(19) United States
(12) Patent Application Publication
(10) Pub. No.: US 2004/0265997 A1
(43) Pub. Date: Dec. 30, 2004

(54) Bio-artificial liver system

(76) Inventor: Sung-Soo Park, Rancho Palos Verdes, CA (US)

Correspondence Address: THE MAXHAM FIRM, 750 "B" STREET, SUITE 3100, SAN DIEGO, CA, 92101, US

(21) Appl. No.: 10/608,372

(22) Filed: Jun. 27, 2003

(51) Int. Cl. C12N 5/08; A61M 31/00

(52) U.S. Cl. 435/370; 604/500

(57) ABSTRACT

A bioartificial liver system has a separator for separating plasma and blood, a liver-slice culture apparatus, or bioreactor, to detoxify the plasma. The bioreactor has a chamber with plasma and gas inlets, at least two meshes mounted parallel one above the another, near the upper portion of the chamber forming at least two horizontal layers separated by a space. A plurality of liver slices positioned within the space, a supply of plasma is provided to the chamber so that it rises to contact the liver slices, and is alternatively removed from contacting the liver slices, and a supply of gas is provided to the top of the chamber. The system also includes a reservoir for containing plasma entering and exiting the chamber. Methods are provided for detoxifying plasma using the bioartificial liver system.

### BACKROUND OF THE INVENTION

1. Field of the Invention

The invention relates to a biological artificial liver system and, more particularly, a bioreactor for blood detoxification and use thereof.

2. Discussion of Related Art

Liver failure, notwithstanding advances in medical management, remains a cause of considerable morbidity and mortality in the developed world. Liver disease itself is a serious problem. It has been estimated that one in ten, or 25 million Americans, are afflicted with liver disease. Each year over 43,000 people die of liver disease in the United States, and hospitalization costs exceed $8 billion.

Cirrhosis is the seventh-leading cause of death and the fourth-leading disease-related cause of death in people between the ages of 25 to 44. Twenty-five thousand people die annually from chronic liver disease and cirrhosis. Ten thousand people die annually from Hepatitis C. Five thousand people die from Hepatitis B, with an estimated new infection rate of 250,000 people annually. One thousand people die from primary liver cancer annually (American Liver Foundation).

Acute episodes of liver failure occur at alarming speed. Because of the life-threatening complications of acute liver failure, 75% of patients die within a few days of onset. Fulminant hepatic failure (FHF) is the severe impairment of hepatic functions in the absence of preexisting liver disease. Usually, the pathogenesis of FHF begins with exposure of a susceptible person to an agent capable of producing severe hepatic injury, although the exact etiology remains unidentified in most cases of FHF. One theory highlights the effect of accumulation of neurotoxic or neuroactive substances as a consequence of hepatocellular failure. These substances include false neurotransmitters, ammonia, increased gamma-aminobutyric acid receptor activity, and increased circulating levels of endogenous benzodiazepine-like substances. Viral agents may cause damage to hepatocytes either by direct cytotoxic effect or as a result of hyperimmune response. Apparently, the interaction between agent and host determines the incidence of FHF. Hepatotoxic metabolites, which accumulate as a result of errors in metabolism or of taking hepatotoxic drugs, may cause injury to the hepatocytes. Serum ammonia levels may be normal or slightly elevated, even in patients who are deeply comatose.

In FHF, hepatic regeneration is usually insufficient to keep the patient alive. Therefore, the only satisfactory treatment for FHF is organ transplantation. Although these operations have a 70-80% five-year survival rate, there is a dramatic shortage of organ donors. For example, there are fewer than 5,000 liver transplants performed each year in the United States (Organ Procurement and Transplantation Network).

Therefore, there is a great need for a temporary liver support to either allow the patient's native liver to regenerate, or as a bridge to organ transplantation. Various forms of temporary liver support include haemodialysis, haemofiltration, exchange transfusion, plasma exchange, resin haemoperfusion, charcoal perfusion, bioartificial liver, extra corporeal liver assist devices, and extracorporeal whole liver perfusion.

There has been a significant amount of work on the development of bioartificial liver devices. It is thought that hepatic function can only be replaced with the biological substrate, that is, liver cells or a whole liver specimen, which requires the availability of liver tissue from xenogenic or human sources. Recent efforts have combined mechanical and biologic support systems in hybrid liver support devices. The mechanical component of these hybrid devices serves both to remove toxins and to create a barrier between the patient's serum and the biologic component of the liver support device. The biologic component of these hybrid liver support devices may consist of liver slices, granulated liver, or hepatocytes from low-grade tumor cells or porcine hepatocytes. These biologic components are housed within bioreactors. However problems remain with respect to maintaining the functionality of the individual cell lines used in these devices. Most devices use immortalized cell lines. It has been found that over time these cells lose specific functions.

There are several groups or companies developing bioartificial livers, for example, Circe Biomedical (Lexington, Mass.), Vitagen (La Jolla, Calif.), Excorp Medical (Oakdale, Minn.), and Algenix (Shoreview, Minn.). The Circe Biomedical device integrates viable liver cells with biocompatible membranes into an extracorporeal, bioartificial liver assist system. Vitagen's ELAD® (Extracorporeal Liver Assist Device) Artificial Liver is a two-chambered hollow-fiber cartridge containing a cultured human liver cell line (C3A). The cartridge contains a semipermeable membrane with a characterized molecular weight cutoff. This membrane allows for physical compartmentalization of the cultured human cell line and the patient's ultrafiltrate. Algenix provides a system in which an external liver support system uses porcine liver cells. Individual porcine hepatocytes pass through a membrane to process the human blood cells. Excorp Medical's device contains a hollow fiber membrane (with 100 kDa cutoff) bioreactor that separates the patient's blood from approximately 100 grams of primary porcine hepatocytes that have been harvested from purpose-raised, pathogen-free pigs. Blood passes though a cylinder filled with hollow polymer fibers and a suspension containing billions of pig liver cells. The fibers act as a barrier to prevent proteins and cell byproducts of the pig cells from directly contacting the patient's blood but allow the necessary contact between the cells so that the toxins in the blood can be removed.

These devices represent an improvement over pre-existing technology, but they still have particular disadvantages. The effectiveness of these devices, all of which use individual hepatocytes, is limited due to the lack of cell to cell interactions, which characterize the liver in its in vivo state. Accordingly, a bioartificial liver with improved efficiency, viability and functionality is desired.

### SUMMARY OF THE INVENTION

It is one object of the invention to provide a bioartificial liver system (BAL) having liver slices maintained in a liver-slice culture, or bioreactor, apparatus.

The present invention provides a bioartificial liver system for treating hepatic functional impairment. The system has a means for separating a blood stream from a patient into plasma and blood cells, a means for detoxifying the plasma. The detoxifying means has a sealable chamber having a plasma inlet and a gas inlet, a plurality of animal liver slices, and a mesh at least partially surrounding the animal liver slices so as to form a space and to retain the slices within this space. The mesh is positioned approximately horizontal at or near an upper portion of the chamber. The system also has a means for selectively supplying and removing plasma from the chamber. This means is configured so that when the plasma is supplied to the chamber the plasma comes into contact with the liver slices, and when the plasma is removed from the chamber the plasma is not in contact with the liver slices. The system also has a means for supplying a gas to the top of the chamber, a reservoir for containing plasma as it enters and exits the chamber, and a means for reintroducing the plasma and blood cells back to the patient. In this system the animal liver slices are cultured in an environment of an oxygenated gas and under the supply of a liquid culture medium so that the slices are exposed alternatively at regular intervals to the medium and to the gas thereby detoxifying the plasma and treating hepatic functional impairment.

The present invention also provides a bioartificial liver system which is composed of a means for separating a blood stream taken from a patient with hepatic functional impairment into a plasma stream and a blood cell stream, and a liver-slice culture apparatus used as a bioreactor to detoxify the plasma stream. The system includes a chamber having an inlet for plasma and an inlet and outlet for a gas, at least two panels with a multiplicity of openings mounted approximately parallel one another near the upper portion of the chamber so as to form at least two layers separated by a space, a plurality of liver slices positioned within the space, means for selectively supplying and removing plasma in the chamber so that the plasma in the chamber comes into contact with the liver slices, and is removed from contact with the liver slices, means for supplying a gas to the top of the chamber, and a reservoir for containing plasma as it enters and exits the chamber. The animal slices are cultured in an environment of an oxygenated gas under the supply of a liquid culture medium at regular intervals so that the slices are exposed alternatively to the medium and to the gas.

In another embodiment of the invention, the system also includes a second reservoir for receiving detoxified plasma from the chamber. In a further embodiment, the gas is a mixture of oxygen and carbon dioxide. In another embodiment the gas-to-plasma ratio, as they are in contact with the liver slices, is about 1:2 to about 1:4. In a preferred embodiment, this exposure-time ratio is about 1:3.

The invention also provides a method to detoxify the plasma of a mammal, the method including separating plasma from whole blood, contacting the plasma with animal liver slices, the animal liver slices being contained in a bioreactor, the bioreactor being made up of a sealable chamber having a plasma inlet and a gas inlet, at least two meshes mounted approximately parallel, one above the other near the upper portion of the chamber so as to form at least two approximately horizontal layers separated by a space, a plurality of animal liver slices positioned within the space, means for selectively supplying and removing plasma in the chamber so that the plasma in the chamber comes into contact with the liver slices, and is removed from contact with the liver slices, means for supplying a gas to the top of the chamber, a reservoir for containing plasma as it enters and exits the chamber, the method further involving contacting the liver slices with a gas mixture of oxygen and carbon dioxide, exposing the liver slices alternatively to plasma and the gas mixture of oxygen and carbon dioxide gas in a ratio of about 1:3, and returning to detoxified plasma to the mammal.

The invention also provides methods to treat a hepatic failure patient using the bio-artificial liver system disclosed herein.

### BRIEF DESCRIPTION OF THE DRAWING

Further particularities and advantages of the invention will become clear from the following description of preferred embodiment, with reference to the drawing, in which:

FIG. 1 is a schematic diagram of the bioartificial liver system of the present invention;

FIG. 2 shows an embodiment of the bioartificial liver system of the present invention;

FIG. 3A is a side sectional view of the liver-slice arrangement of the present invention;

FIG. 3B is a perspective view of the liver-slice arrangement of the present invention;

FIG. 4A is a graphical representation of in vitro lidocaine clearance using the bioartificial liver system of the present invention;

FIG. 4B is a graphical representation of in vitro lidocaine clearance using the bioartificial liver system of the present invention;

FIG. 5 is a graphical representation of in vitro DMX concentration using the bioartificial liver system of the present invention; and

FIG. 6 is a graphical representation of in vitro ammonia clearance using the bioartificial liver system of the present invention.

### DETAILED DESCRIPTION OF THE PREFERRED EMBODIMENTS

In accordance with the present invention, there is provided a bioartificial liver system for treating a hepatic failure patient. The system has a separator for separating a blood stream taken from the patient into a plasma stream and a blood cell stream, and a liver-slice culture apparatus used as a bioreactor to detoxify the plasma stream.

As used herein, the term “detoxification” or “detoxify” refers to reducing the effect of or removing a harmful or poisonous substance from a patient's blood or plasma. For example, as contemplated herein, ammonia is a substance which can be detoxified using the present invention. If ammonia is allowed to build up, it will eventually reach toxic or poisonous levels in the patient. Ammonia has been established to play a role in the development of hepatic encephalopathy.

In the present invention the culture apparatus has a chamber having a plasma inlet and a gas inlet, at least two meshes mounted parallel, one above the other near the upper portion of the chamber so as to form at least two horizontal layers separated by a space. A plurality of liver slices is positioned within this space. There is a means for supplying plasma to the chamber so that the plasma in the chamber rises to come into contact with the liver slices. This means is also able to remove the plasma from contact with the liver slices. There is also a means for supplying a gas to the top of the chamber so that the liver slices are exposed alternatively to gas and plasma. Additionally, a reservoir is provided for containing plasma as it enters and exits the chamber. The chamber is preferably thermoregulated.

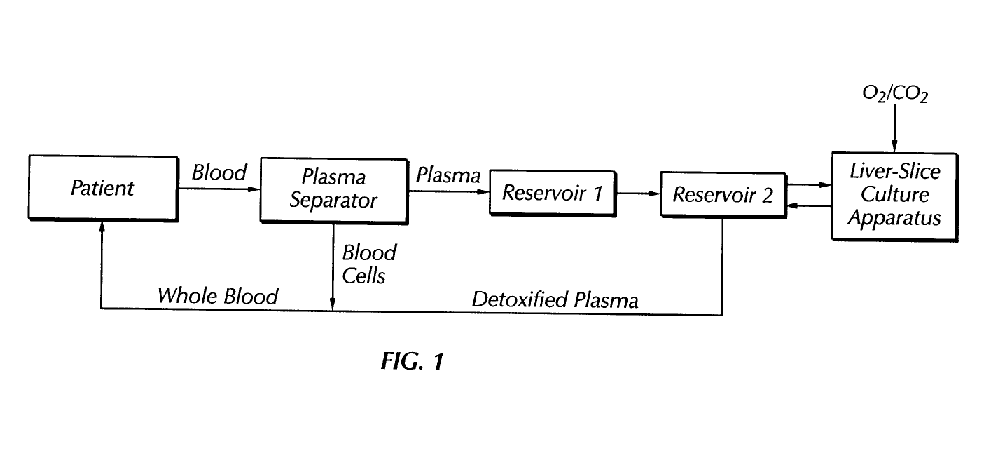

FIG. 1 is a schematic representation of the bioartificial liver system in accordance with the present invention. Blood is drawn from the patient into a plasma separator, the specifics of which are well known to those skilled in the art. Plasma and blood cells are separated and the plasma is introduced into a first reservoir. Plasma then enters a second reservoir. From the second reservoir the plasma is introduced into the liver-slice culture apparatus, or bioreactor. Liver slices are arranged between two wire meshes and placed at or near the top section of the bioreactor. As plasma is introduced into the bioreactor, the fluid level begins to rise until it comes into contact with the liver slices at or near the top of the bioreactor.

Oxygenated gas is introduced in the top of the bioreactor. The gas is preferably a mixture of 95% by volume O₂ and 5% by volume CO₂, and is supplied at a pressure ranging from 1 to 10 ATM to the chamber through a gas inlet and discharged therefrom through a gas outlet, while controlling the pressure by a pressure controller. A solenoid valve may be coupled with the pressure controller to maintain a pre-set gas pressure. A gas sterilizing device, for example, a syringe filter having a pore size of about 0.22 μm, is preferably installed in the gas inlet line to filter out microbes, thereby sterilizing the supply gas to the chamber. Another gas sterilizing device is preferably installed in the gas outlet in order to prevent backflow of microbes in the atmospheric gas.

Stabilization of the liver slices is an important feature of the invention. The liver slices are cultured under the supplies of liquid culture medium and an oxygenated gas. The liquid culture medium, or the plasma, is supplied through the reservoir into the bioreactor and the oxygenated gas is supplied through the top of the bioreactor. Each are supplied at regular intervals so that each of the liver slices is exposed alternately to the medium and the gas at an exposure-time ratio ranging from about 1:2 to about 1:4, preferably about 1:2.5 to about 1:3.5, and most preferably about 1:3.

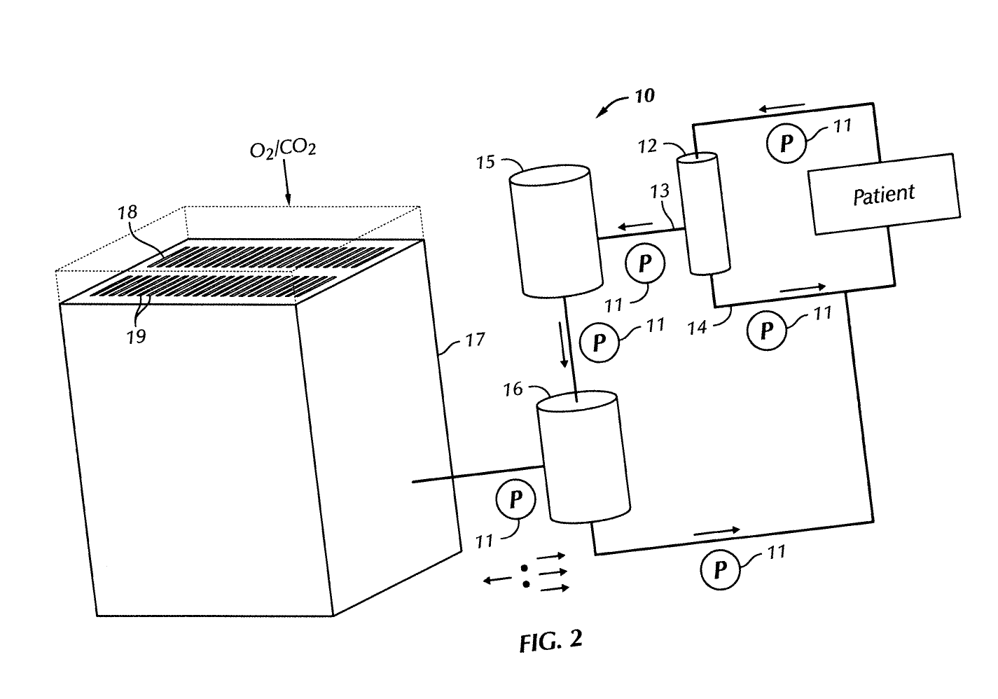

Referring now to the drawing, and more particularly FIG. 2, there is shown an embodiment of the artificial liver system of the present invention. The bioartificial liver system is represented generally by numeral 10. A patient in need of blood detoxification is connected to plasma separator 12. The patient's blood stream is sent to the plasma separator to obtain plasma stream 13 and blood cell stream 14. The plasma stream is led to first plasma reservoir 15 and then to second plasma reservoir 16. The controlled movement of plasma is regulated by pumps 11 positioned throughout the system as indicated in FIG. 2. The plasma enters bioreactor 17 and continues to rise to a desired level. Near the top portion of the bioreactor is the liver-slice apparatus 18. The liver-slice apparatus contains a plurality of liver-slices 19 held between two meshes. The liver-slice apparatus is positioned approximately horizontally. Oxygenated gas is introduced from the top of the bioreactor. The plasma and gas flow are therefore controlled so that the liver slices do not suffer from necrosis due to an insufficient oxygen or plasma supply.

The detoxified plasma stream is collected at the bottom of the bioreactor and is pumped back out of the bioreactor into plasma reservoir 16. From there the detoxified plasma is recombined with blood cell stream 14 emanating from the plasma separator and returned to the patient.

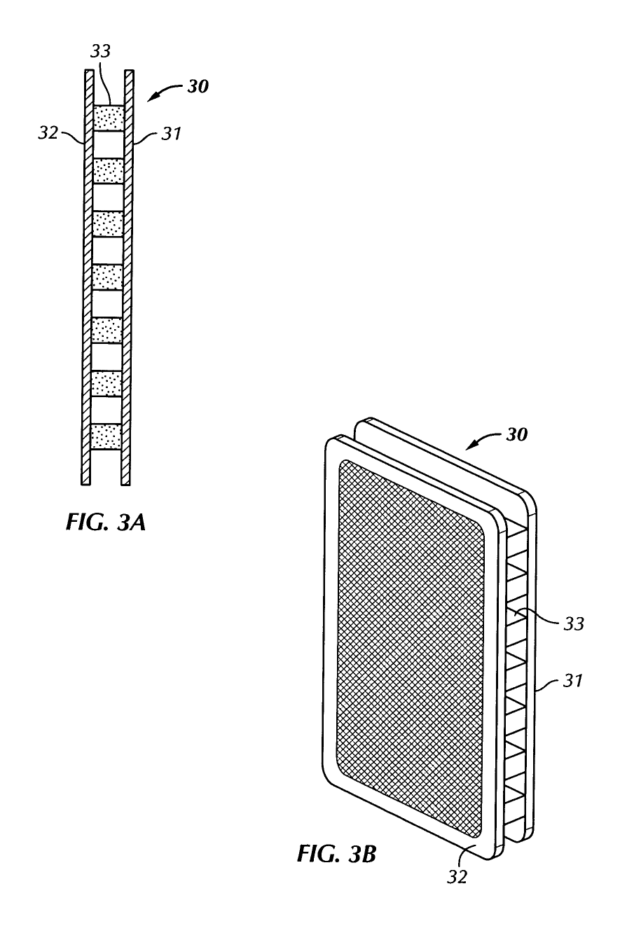

FIGS. 3A and 3B show the liver-slice apparatus of the present invention, as represented by numeral 30. Two stainless steel meshes 31 and 32 are provided, the size of which can be chosen based on the dimensions of the bioreactor. These two meshes are preferably arranged in parallel. In a preferred embodiment, the meshes have about a 0.26 mm pore size. Also, in a preferred embodiment, the meshes are pressed to ensure consistent flatness. Between meshes 31 and 32 are a plurality of liver-slices 33 arranged in an orderly fashion. The two meshes are positioned on each side of the liver slices with enough room so as to not crush the liver slices, but also so as to hold them sufficiently so that they do not get washed away by the plasma. Although FIGS. 3A and 3B show a relatively small number of liver slices positioned between the meshes, it is to be understood that the efficiency of the apparatus is dependent upon the number of liver slices employed. In addition, although two meshes are shown, it is contemplated herein that a single mesh may be used. That mesh is formed to surround, at least partially, the liver slices thereby forming a space and to retain them in that space. For example, the mesh could be formed in a suitably dimensioned U-shape.

Liver slices used in the present invention may be obtained from a suitable animal, for example, a rabbit, pig, dog or human, depending on the intended use of the bioartificial liver. Also, they may be of any size or shape suitable for maintaining the viability and essential functions thereof. In the present invention the liver slices are preferred to have a thickness ranging from about 10 μm to about 2,000 μm, and more preferably ranging from about 100 μm to about 500 μm.

The present invention is much more efficient at detoxifying the blood of a hepatic failure patient than a system employing isolated hepatocytes. This is shown through the following examples.

### EXAMPLE 1

### Recovery from Hepatic Failure

Eleven Mongol dogs, weighing between 22-25 kg, were anesthetized with 15 mg dose of Ketamine, and 0.5 g D-galactosamine/kg was intravenously injected in the brachial vein. When coma was observed, cannulation of the femoral artery and vein for plasma separation was performed. Rabbit liver slices 260 μm thick (1.8×10⁶ hepatocytes) were plated into the bioreactor described above. Thirty to thirty-five hours after administration, the dogs showed abnormal behavior, and seizure and rapid progression to coma were observed. Blood chemistry (glucose, ammonia, fibrinogen, LDH) and histopathology were assessed in each animal. The results are summarized in Tables 1 and 2 below.

TABLE 1

| | Ammonia | AST | ALT | LDH |
|---|---|---|---|---|
| | (μg/dL) | (IU/L) | (IU/L) | (IU/L) |
| Coma | 648 ± 213 | 5701 ± 2979 | 7585 ± 2459 | 1971 ± 133 |
| 1 hour of BAL | 327 ± 70 | 5612 ± 2969 | 6982 ± 2870 | 1719 ± 249 |

The results in Table 1 show that after 1 hour of BAL treatment, all blood levels returned to relatively normal ranges. In fact, after 1 hour of BAL treatment, full recovery of animal activity was observed.

TABLE 2

| | BAL Treated Group | | Control Group | |
|---|---|---|---|---|
| | Plasma Glucose | Fibrinogen | Plasma Glucose | Fibrinogen |
| | (mg/dL) | (mg/dL) | (mg/dL) | (mg/dL) |
| Coma | 96 ± 8 | 273 ± 116 | 96 ± 11 | 378 ± 54 |
| 1 hour | 36 ± 9 | 295 ± 119 | 33 ± 5 | 75 ± 39 |
| 2 hours | 31 ± 8 | 238 ± 87 | 9 ± 6 | 70 ± 43 |
| | | | (expired) | (expired) |

All animals in the control group (n=3) developed coma and expired within 2 hours. At autopsy of the control group, all livers showed total hepatic necrosis. By contrast all three BAL treated animals survived until 6 hours.

The results demonstrate that the bioartificial liver performs much like a normal liver in vivo when the animal derived plasma flows through the slice culture device. The present invention is unique in utilizing liver slices instead of hepatocytes. The data suggests that a slice culture system can be successfully applied to a bioartificial liver.

### EXAMPLE 2

### In Vitro Performance

The following example illustrates the in vitro performance of a flat plate bioreactor using liver slices and forms the model for the bioartificial liver device of the present invention. The example here shows the efficiency of liver slices to metabolize ammonia and lidocaine.

The liver converts ammonia to urea, which is excreted into the urine by the kidneys. In the presence of severe liver disease, ammonia accumulates in the blood because of both decreased blood clearance and decreased ability to form urea. Elevated ammonia levels can be toxic, especially to the brain, and play a role in the development of hepatic encephalopathy. Accordingly, liver function can be assessed by measuring ammonia clearance.

In addition, lidocaine is a drug that can be converted by the liver from a toxic form into a non-toxic metabolite known as dimethyl xylidine (DMX). The measure of lidocaine clearance is an indication of the performance of the liver.

A 3 to 3.5 kg rabbit was euthanized and liver slices obtained. The slices were approximately 1 cm in diameter with an average weight of 50 mg. Approximately 2 grams total were used in this example. The slices were then pre-cultured by immersion in approximately 200 ml of Williams E media with 10% FCS and drained upon exposure to an oxygenated gas. Each liver slice is exposed alternately to the medium and gas at an exposure-time ratio of approximately 1:3.

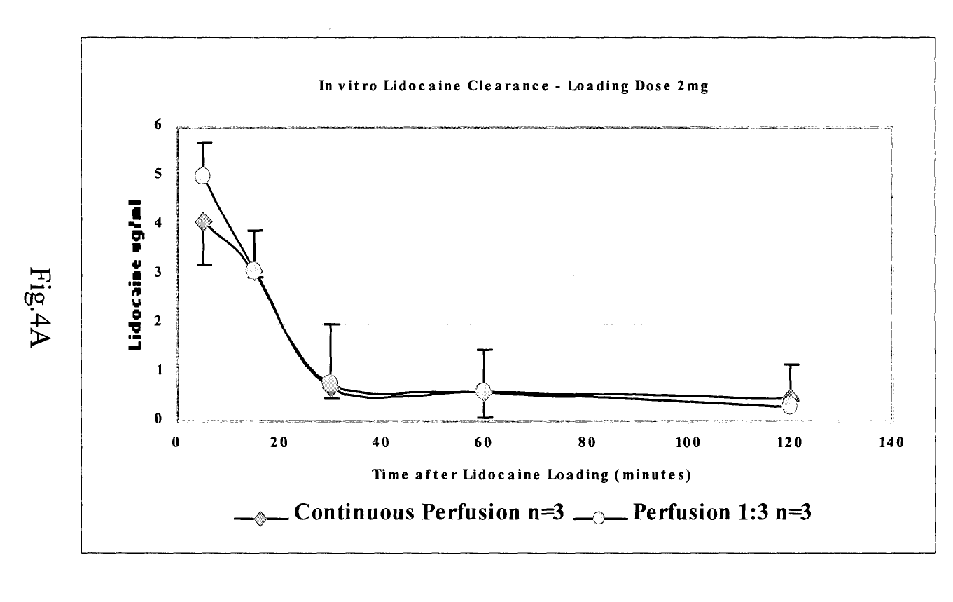

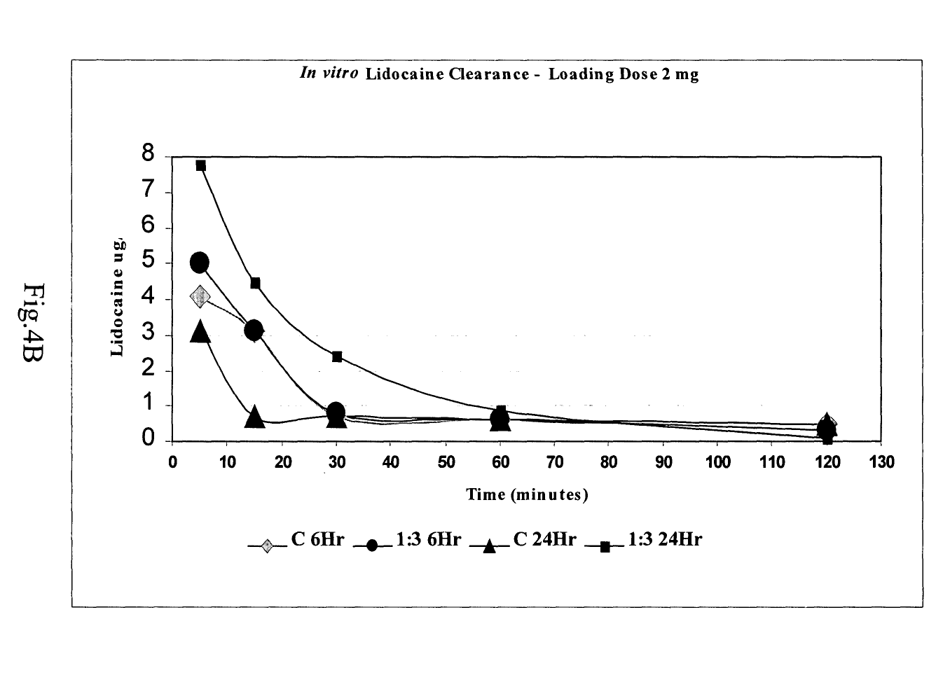

The gas mixture, approximately 95% oxygen, 5% CO₂ at 1 ATM was maintained in the chamber throughout the study. The gas mixture was exchanged every twelve minutes. Bolus doses of lidocaine (2 mg) or ammonia (20 mg) were injected. The ammonia and DMX were measured by collecting samples at 0, 5, 15, 30, 60, 90 and 120 minutes, after 6 hours and 25 hours of cultivation. The results are summarized in FIGS. 4A, 4B, 5 and 6.

FIG. 4A is a graphical representation of in vitro clearance of a 2 mg dose of Lidocaine. Continuous perfusion was performed (as indicated by the diamonds) and intermittent perfusion (time-exposure ratio of 1:3) was also performed (indicated by the circles). Three separate trials were performed for each. At approximately 30 minutes after lidocaine loadine, the level of lidocaine dropped from between 3.2 and 5.8 μg to approximately 0.9 μg. This level was reduced to approximately 0.5 μg at 120 minutes. The results demonstrate that the device of the present invention reduced lidocaine levels to non-toxic levels within 30 minutes. As compared to continuous medium perfusion, the intermittent perfusion (approximately 1:3) requires less volume of medium while achieving substantially the same results.

FIG. 4B is a graphical representation of in vitro clearance of a 2 mg dose of Lidocaine for exposure times of 6 hours and 24 hours. Initial readings of Lidocaine were between 2 μg and 7.8 μg. However, within 30 minutes Lidocaine levels reduced to approximately 0.80 μg for the 6 hour trials and for the continuous perfusion 24 hour trial. Within 60 minutes all trials were showing lidocaine levels between 0.75 μg and 1 μg. Again, the results demonstrate the efficiency of the bioreactor to reduce lidocaine levels to non-toxic levels with intermittent perfusion.

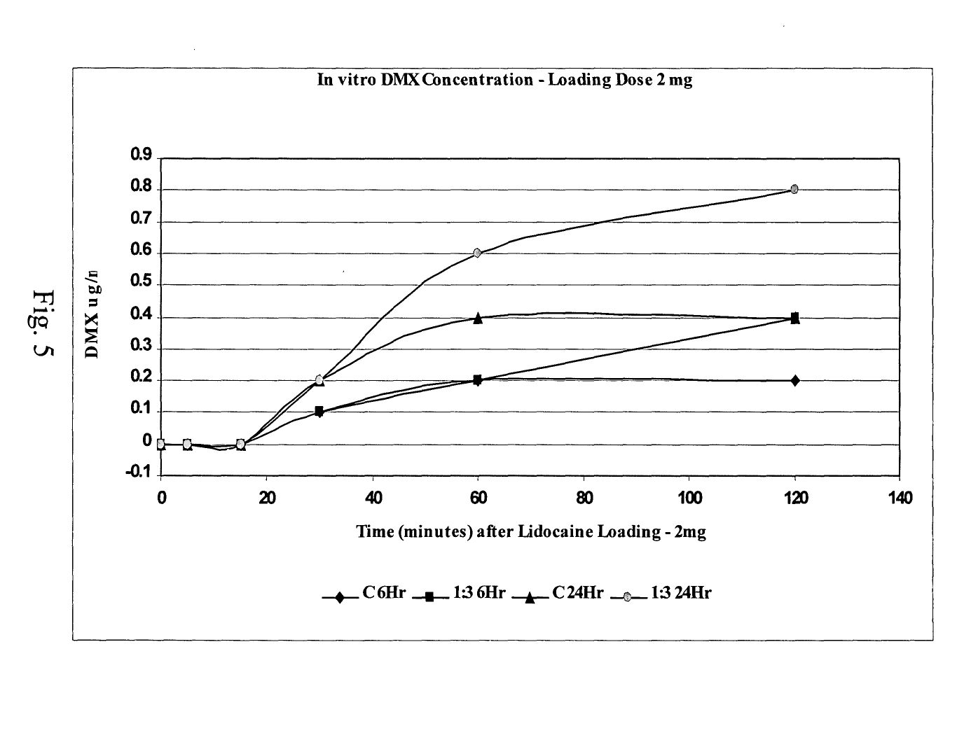

FIG. 5 is a graphical representation of in vitro DMX concentration build-up from a 2 mg Lidocaine dose. Initially DMX concentration remained approximately zero, until approximately 18 minutes. The DMX metabolites were observed increasing in concentration after 18 minutes and reached approximately maximal values at 60 minutes. However, for the 24 hour 1:3 exposure trial, the DMX concentration continued to increase up to 120 minutes. These results show the ability of the present invention to metabolize lidocaine (as indicated by the DMX metabolite concentration increasing over time). At approximately 60 minutes maximal DMX concentration was observed. There was no significant difference between the continuous perfusion trial and the intermittent perfusion trial, except for the 24 hour exposure trial mentioned above.

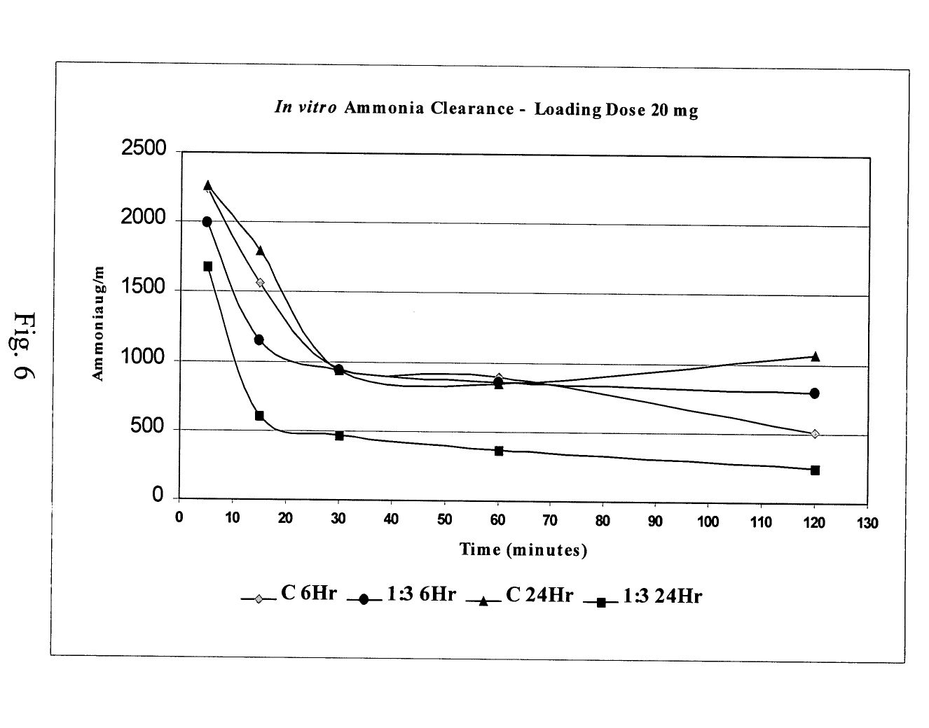

FIG. 6 is a graphical representation of in vitro ammonia clearance of a 20 mg loading dose. At approximately 30 minutes maximal ammonia clearance was observed in all trials. These results demonstrate the ability of the present invention to remove ammonia relatively quickly to non-toxic levels. In addition, there was no significant difference between the trials with continuous perfusion and those with intermittent perfusion, thereby indicating that less medium can be used while still retaining activity and efficiency of the device.

While the present invention has been illustrated and described by means of a specific embodiment, it is to be understood that numerous changes and modifications can be made therein without departing from the spirit and scope of the invention.

What is claimed is:

1. A bioartificial liver system for treating hepatic functional impairment, said system comprising:
a means for separating a blood stream from a patient into plasma and blood cells;
a means for detoxifying the plasma, said means comprising:
a sealable chamber having a plasma inlet and a gas inlet;
a plurality of animal liver slices; and
a mesh at least partially surrounding said animal liver slices so as to form a space and to retain said slices within said space, said mesh being positioned approximately horizontal at or near an upper portion of the chamber;
a means for selectively supplying and removing plasma from the chamber, said means being configured so that when the plasma is supplied to the chamber the plasma comes into contact with the liver slices, and when the plasma is removed from the chamber the plasma is not in contact with the liver slices;
a means for supplying a gas to the top of the chamber;
a reservoir for containing plasma as it enters and exits the chamber; and
a means for reintroducing the plasma and blood cells back to the patient,
wherein said animal liver slices are cultured in an environment of an oxygenated gas and under the supply of a liquid culture medium so that said slices are exposed alternatively at regular intervals to said medium and to said gas thereby detoxifying the plasma and treating hepatic functional impairment.

2. The system of claim 1, the culture apparatus further comprising a second reservoir for receiving detoxified plasma from the chamber.

3. The system of claim 1, wherein the gas is a mixture of oxygen and carbon dioxide.

4. The system of claim 3, wherein the gas-to-plasma exposure time ratio to the animal liver slices is about 1:2 to about 1:4.

5. The system of claim 3, wherein the gas-to-plasma exposure time ratio to the animal liver slices is about 1:3.

6. The system of claim 2, further comprising an immunological filter inserted downstream from the second reservoir.

7. The system of claim 1, wherein the chamber is thermoregulated.

8. A bioartificial liver system for treating a patient with hepatic functional impairment, said system comprising:
a means for separating a blood stream taken from the patient into a plasma stream and a blood cell stream; and
a liver-slice culture apparatus used as a bioreactor to detoxify the plasma stream, the culture apparatus comprising:
a sealable chamber having a plasma inlet and a gas inlet;
at least two meshes mounted approximately parallel, one above the other, near the upper portion of the chamber so as to form at least two approximately horizontal layers separated by a space;
a plurality of animal liver slices positioned within said space;
means for selectively supplying and removing plasma in the chamber so that the plasma in the chamber comes into contact with the liver slices, and is removed from contact with the liver slices;
means for supplying a gas to the top of the chamber; and
a reservoir for containing plasma as it enters and exits the chamber, said animal liver slices being cultured in an environment of an oxygenated gas under the supply of a liquid culture medium at regular intervals so that said slices are exposed alternatively to the medium and to the gas.

9. The system of claim 8, the culture apparatus further comprising a second reservoir for receiving detoxified plasma from the chamber.

10. The system of claim 8, wherein the gas is a mixture of oxygen and carbon dioxide.

11. The system of claim 10, wherein the gas-to-plasma exposure time ratio to the animal liver slices is about 1:2 to about 1:4.

12. The system of claim 10, wherein the gas-to-plasma exposure time ratio to the animal liver slices is about 1:3.

13. The system of claim 9, further comprising an immunological filter inserted downstream from the second reservoir.

14. The system of claim 8, wherein the chamber is thermoregulated.

15. A method to detoxify the plasma from a mammal, the method comprising:
separating plasma from whole blood of the mammal;
contacting the plasma with animal liver slices, the animal liver slices being contained in a bioreactor, the bioreactor being made up of a sealable chamber having a plasma inlet and a gas inlet, at least two meshes mounted approximately parallel, one above the other near the upper portion of the chamber so as to form at least two approximately horizontal layers separated by a space, a plurality of animal liver slices positioned within said space, means for selectively supplying and removing plasma in the chamber so that the plasma in the chamber comes into contact with the liver slices, and is removed from contact with the liver slices, means for supplying a gas to the top of the chamber, a reservoir for containing plasma as it enters and exits the chamber the space between two meshes, the method further comprising the steps of:
contacting the liver slices with a gas mixture of oxygen and carbon dioxide;
exposing the liver slices alternatively to plasma and the gas mixture of oxygen and carbon dioxide gas; and
returning detoxified plasma to the mammal.

16. The method of claim 15, wherein the gas-to-plasma exposure time ratio to the animal liver slices is about 1:3

17. A method of treating a hepatic failure patient, the method comprising:
separating the plasma from the whole blood of the mammal;
contacting the plasma with animal liver slices contained in a space between two meshes;
contacting the liver slices with a gas mixture of oxygen and carbon dioxide;
exposing the liver slices alternatively to the plasma and the gas mixture of oxygen and carbon dioxide gas; and
returning to detoxified plasma to the mammal and the mammal's blood is thereby detoxified.

18. The method of claim 17, wherein the gas-to-plasma exposure time ratio to the animal liver slices is about 1:3.
